(19) United States
(12) Patent Application Publication
(10) Pub. No.: US 2013/0030319 A1
(43) Pub. Date: Jan. 31, 2013

(54) CARDIAC MONITORING USING SPINAL CORD STIMULATION ELECTRODES

(71) Applicants: Douglas A. Hettrick, Andover, MN (US); Pascalle Reiters, Gronsveld (NL); Shantanu Sarkar, Roseville, MN (US); Todd M. Zielinski, Ham Lake, MN (US)

(72) Inventors: Douglas A. Hettrick, Andover, MN (US); Pascalle Reiters, Gronsveld (NL); Shantanu Sarkar, Roseville, MN (US); Todd M. Zielinski, Ham Lake, MN (US)

(73) Assignee: Medtronic, Inc., Minneapolis, MN (US)

(21) Appl. No.: 13/562,112

(22) Filed: Jul. 30, 2012

Related U.S. Application Data
(60) Provisional application No. 61/512,967, filed on Jul. 29, 2011.

(51) Int. Cl. A61B 5/053

(52) U.S. Cl. 600/547

(57) ABSTRACT

Methods and systems for use in cardiac monitoring may periodically measure intrathoracic impedance using one or more pairs of electrodes and analyze measured impedance values to identify changes in the patient's thoracic fluid content for use in cardiac monitoring. The intrathoracic impedance is measured using at least one spinal cord stimulation electrode.

### CROSS-REFERENCE TO RELATED APPLICATIONS

This application claims the benefit of U.S. Provisional Application No. 61/512,967, filed on Jul. 29, 2011. The disclosure of the above application is incorporated herein by reference in its entirety.

### BACKGROUND

The disclosure herein relates to cardiac monitoring using spinal cord stimulation electrodes, and further to devices for performing and implementing such methods.

Spinal cord stimulation (SCS), which may be generally described as electrical stimulation applied to the central nervous system in the spinal cord area, may be used to perform cardiac chronic pain therapy and acute angina therapy among other therapies (e.g., pain management, etc.). For example, some pre-clinical data has suggested that SCS may influence the autonomic nervous system (ANS), which may positively affect heart failure status (see, e.g., Lopshire et al. Spinal Cord Stimulation Improves Ventricular Function and Reduces Ventricular Arrhythmias in a Canine Postinfarction Heart Failure Model. Circulation 2009; 120: 286-294). Further, for example, SCS may be used to restore autonomic balance in case of angina, heart failure, and arrhythmias.

SCS has also been shown to improve cardiac contractibility, to further improve the pressure-volume relationship within the heart, and to reduce sympathetic activity of the cardiac tissue to reduce the likelihood of ventricular arrhythmias. The electrical stimulation delivered by SCS may produce effects and provide benefits through physiological mechanism similar to those induced by prescription beta-blocker drugs, which have been shown to vasodilate peripheral arterioles and increase blood flow to the limbs. SCS may further cause the production of neuropeptides such as CGRP, NO, and VIP that are known vasodilators, which may assist in redirection of blood flow from regions of high flow to regions of low flow and improve the efficiency of the heart. In ischemic dilated cardiomyopathy patients, SCS may suppress or reduce subendocardial ischemia, and hence be cardio-protective. SCS may further result in improvements to the operational efficiency and function of cardiac tissue even in the presence of reduced blood supply.

A SCS system generally includes a plurality of electrodes located on at least one electrode lead. The electrode lead is implanted in a patient such that the electrodes are located proximate (e.g., along) the spinal cord (e.g., the dura of the spinal cord). Electrical stimulation (e.g., electrical pulses) may then be delivered through the electrodes to the nerve fibers within the spinal cord for therapeutic effect.

Some modern implantable cardiac therapy devices including implantable cardioverter-defibrillators (ICD) and cardiac resynchronization therapy (CRT) devices often provide additional monitoring capabilities (e.g., in addition to their therapeutic capabilities) that provide information on the physical state of the patient. For example, implantable devices may chronically monitor heat rate, heart variability, patient activity levels as well as the timing duration, and severity of both atrial and ventricular arrhythmias. The diagnostic parameters may then be stored in the computer memory of the device and may be retrieved by a clinician through telemetric interfaces either directly or remotely. In particular, an index of potentially worsening heart failure may also be provided. This index may be based on the theory that pulmonary congestion and edema secondary to acutely worsening heart failure is associated with measureable decreases in intrathoracic impedance. An impedance based fluid index parameter has been shown to be clinically useful in multiple clinical trials (see, e.g., Yu CM, Wang L, Chau E, et al. Intrathoracic impedance monitoring in patients with heart failure: correlation with fluid status and feasibility of early warning preceding hospitalization. Circulation. Aug. 9, 2005;112(6):841-848 [MID-Heft]; Abraham W T. Superior performance of intrathoracic impedance-derived fluid index versus daily weight monitoring in heart failure patients. Results of the Fluid Accumulation Status Trial. Late Breaking Clinical Trials. J Card Fail. Vol. 15 No. 9 2009, p 813 [FAST]; Whellan D J, Al-Khatib S M, Kloosterman E M, et al. Changes in intrathoracic fluid index predict subsequent adverse events: Results of the multi-site program to access and review Trending Information and Evaluate Correlation to Symptoms in Patients with Heart Failure (PARTNERS H F) Trial. J Card Fail. 2008; 14(9):799 [PARTNERS H F]; and Small R S, Wickemeyer W, Germany R, et al. Changes in intrathoracic impedance are associated with subsequent risk of hospitalizations for acute decompensated heart failure: clinical utility of implanted device monitoring without a patient alert. J Card Fail. August 2009; 15(6):475-481 [OFFISER], each of which are incorporated by reference herein in their entireties).

### SUMMARY

Exemplary methods and systems described herein may use spinal cord stimulation (SCS) electrodes to periodically measure intrathoracic impedance, and then analyze the intrathoracic impedance to identify changes in the patient's thoracic fluid content (e.g., fluid accumulation) for use in cardiac monitoring. In essence, the exemplary methods and systems may provide heart failure monitoring using intrathoracic impedance measured using a therapeutic SCS system.

In at least one embodiment, the exemplary methods described herein may perform one or more impedance measurements using existing SCS electrodes and case electrodes common in an SCS system. The impedance measurements could be used to monitor respiration rate (and hence apnea), peripheral fluid status (e.g., mean impedance trends, etc.), or as a surrogate for pulsatile changes in arterial blood pressure as a surrogate of perfusion. In another embodiment, measurements could be combined with other measurements performed by peripheral devices (e.g., scales, blood pressure cuffs, etc.), and/or other implanted devices (REVEAL devices, etc.) using established artificial intelligence techniques to determine a dynamic heart failure risk score. For example, one or more techniques to determine a dynamic heart failure risk score may be described in U.S. Pat. App. Pub. No. 2010/0030293 A1 filed on Jul 31, 2008 and entitled “Using Multiple Diagnostic Parameters For Predicting Heart Failure Events” to Sarkar et al., which is incorporated by reference herein in its entirety.

One exemplary system for use in cardiac monitoring described herein may include spinal cord stimulation (SCS) apparatus and a control module. The SCS apparatus may include a plurality of SCS electrodes locatable along a patient's spinal cord. The control module may be coupled to the SCS apparatus. The control module may be configured to periodically measure intrathoracic impedance using one or more pairs of SCS electrodes selected from the plurality of SCS electrodes over an extended period of time (e.g., simultaneously measuring intrathoracic impedance a selected number of times throughout each entire day using multiple pairs of SCS electrode over an extended period of time, which may be greater than a day), and generate, for selected time periods (e.g., a day) during the extended period of time, intrathoracic impedance values of the patient based on the periodically measured intrathoracic impedance. The control module may further analyze the intrathoracic impedance values generated for the selected time periods over the extended period of time to identify changes in the patient's thoracic fluid content for use in cardiac monitoring (e.g., changes in the patient's thoracic fluid content may indicate heart failure, etc.).

Another exemplary system for use in cardiac monitoring described herein may include a plurality of SCS electrodes locatable along a patient's spinal cord, at least one supplemental electrode locatable proximate the patient's thoracic cavity, and a control module coupled to the SCS electrodes and the at least one supplemental electrode. The control module may be configured to periodically measure intrathoracic impedance using one or more pairs of electrodes over an extended period of time (e.g., simultaneously measuring intrathoracic impedance a selected number of times throughout each entire day using multiple pairs of electrodes). Each pair of electrodes may include an SCS electrode selected from the plurality of SCS electrodes and an electrode selected from the at least one supplemental electrode. The control module may be further configured to generate, for selected time periods during the extended period of time, intrathoracic impedance values of the patient based on the periodically measured intrathoracic impedance, and analyze the intrathoracic impedance values generated for the selected time periods over the extended period of time to identify changes in the patient's thoracic fluid content for use in cardiac monitoring.

One exemplary method for use in cardiac monitoring using spinal cord stimulation (SCS) apparatus described herein includes providing a plurality of SCS electrodes located along a patient's spinal cord and periodically measuring intrathoracic impedance using one or more pairs of SCS electrodes selected from the plurality of SCS electrodes over an extended period of time (e.g., simultaneously measuring intrathoracic impedance a selected number of times throughout each entire day using multiple pairs of SCS electrode). The exemplary method further includes generating, for selected time periods during the extended period of time, intrathoracic impedance values of the patient based on the periodically measured intrathoracic impedance and analyzing the intrathoracic impedance values generated for the selected time periods over the extended period of time to identify changes in the patient's thoracic fluid content for use in cardiac monitoring.

Another exemplary method for use in cardiac monitoring using spinal cord stimulation (SCS) apparatus described herein includes providing a plurality of SCS electrodes located along a patient's spinal cord, providing at least one supplemental electrode located proximate the patient's thoracic cavity, and periodically measuring intrathoracic impedance using one or more pairs of electrodes over an extended period of time (e.g., simultaneously measuring intrathoracic impedance a selected number of times throughout each entire day using multiple pairs of electrodes). Each pair of electrodes may include an SCS electrode selected from the plurality of SCS electrodes and an electrode selected from the at least one supplemental electrode. The exemplary method may further include generating, for selected time periods during the extended period of time, intrathoracic impedance values of the patient based on the periodically measured intrathoracic impedance and analyzing the intrathoracic impedance values generated for the selected time periods over the extended period of time to identify changes in the patient's thoracic fluid content for use in cardiac monitoring.

In one or more exemplary systems and methods described herein, periodically measuring intrathoracic impedance using one or more pairs of SCS electrodes may include measuring intrathoracic impedance a selected number of times throughout each entire day, generating intrathoracic impedance values of the patient based on the periodically measured intrathoracic impedance may include generating an intrathoracic impedance composite value based at least on multiple intrathoracic impedance measurements taken throughout each entire day, and/or analyzing the intrathoracic impedance values generated for the selected time periods over the extended period of time may include analyzing the intrathoracic impedance composite value generated for each day over the extended period of time (e.g., where the extended period of time is greater than one day).

In one or more exemplary systems and methods described herein, analyzing the intrathoracic impedance values generated for the selected time periods over the extended period of time may include comparing an intrathoracic impedance value for a current selected time period to a baseline value. The baseline value may be at least based on previously generated intrathoracic impedance values.

At least one exemplary system described herein further includes an implantable medical device and at least one exemplary method described herein further includes providing an implantable medical device. The implantable medical device may include a housing and the at least one supplemental electrode may be located on the housing of the implantable medical device. For example, the implantable medical device may be a SCS stimulator for delivering stimulation pulses to one or more of the plurality of SCS electrodes or may be a different implantable medical device separate from an SCS stimulator.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a diagram of an exemplary system including an implantable medical device (IMD).

FIG. 2 is a block diagram of the IMD of FIG. 1.

FIG. 3 depicts various impedance vectors that, e.g., may be used by the system of FIG. 1.

FIG. 4 is a flow chart of an exemplary method for use in cardiac monitoring using SCS apparatus, e.g., using the system of FIG. 1.

FIG. 5 is a graphical depiction of an exemplary method of providing a heart failure risk score, e.g., using the system of FIG. 1 and/or method of FIG. 4.

### DETAILED DESCRIPTION OF EXEMPLARY EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

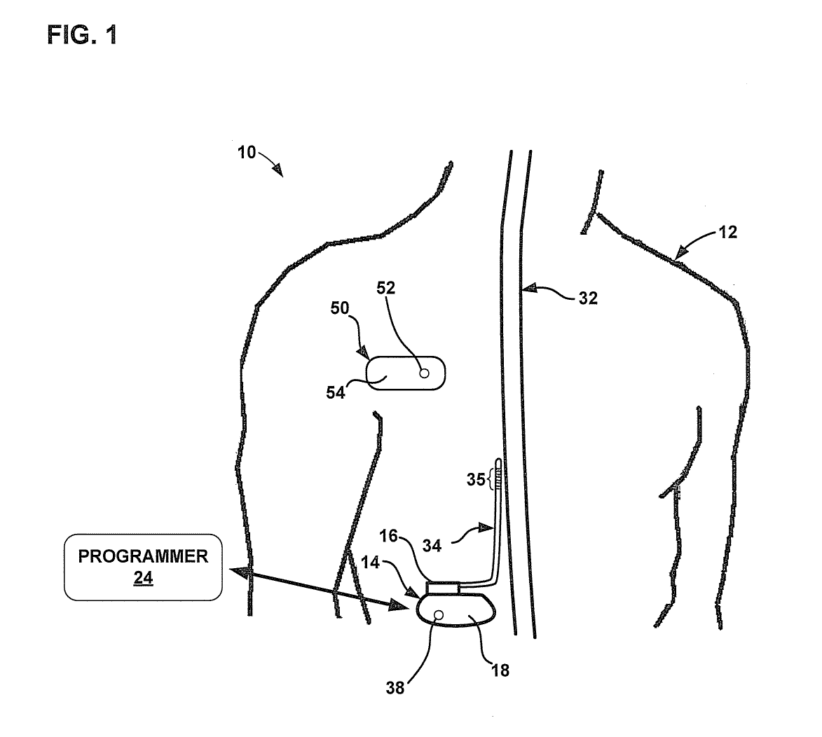

Exemplary systems and methods shall be described with reference to FIGS. 1-5. It will be apparent to one skilled in the art that elements or processes from one embodiment may be used in combination with elements or processes of the other embodiments, and that the possible embodiments of such systems and methods using combinations of features set forth herein is not limited to the specific embodiments shown in the figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

Correlations have been identified between morphometry of real time arterial pressure recorded in the femoral artery and bipolar admittance recorded outside the thoracic cavity in the subcutaneous muscle of the neck near the carotid artery (e.g., from a chronically instrumented dog). Such data demonstrated that it is feasible to obtain clinically useful hemodynamic data using impedance vectors that do not necessarily encompass the lungs. Low frequency variations in admittance due to respiration were also apparent.

Impedance arrays located outside the thoracic cavity may provide impedance signals with potentially useful pulsatile information that resembles arterial pressure. For example, a spinal cord stimulation device and electrodes may be used to provide clinically useful impedance waveforms for the purpose of monitoring heart failure, despite not necessarily encompassing the lungs in the measurement field.

The exemplary systems and methods described herein may provide early warning of fluid accumulation or dehydration in the thorax, most often as a result of cardiac decompensation during heart failure, and may provide guidance to physicians or nurses to titrate medications like diuretics and beta blockers in heart failure patients. Patients with heart failure often live in a delicate balance. Accumulation of fluid can result in frequent and lengthy hospitalizations. Medications can be effective in reducing the accumulation of fluids, but to date there is no accurate, minimally invasive metric of fluid accumulation.

FIG. 1 is a conceptual diagram of an exemplary system 10 that may be used in the delivery of spinal cord stimulation (SCS) therapy to a patient 12 and in the monitoring of intrathoracic impedance of the patient 12. The patient 12 may, but not necessarily, be a human. The exemplary therapy system 10, as shown, may include an implantable medical device 14 (IMD) that is implanted in the patient 12 and includes a least one SCS spinal cord lead 34 that is implanted such that its electrodes are located adjacent the patients spinal cord 32, for example, in the epidural or intrathecal space, between C6 and T4 (e.g., in the vicinity of T4, in the vicinity of T1, etc.).

In at least one embodiment, the lead 34 may be of the type described in U.S. Pat. No. 4,549,556 issued to Tarjan et al. or in commonly assigned U.S. Pat. No. 5,255,691 issued to Otten, U.S. Pat. No. 4,044,774 issued to Corbin et al. or U.S. Pat. No. 5,360,441 issued to Otten, which are all incorporated herein by reference in their entireties, or may correspond to commercially available spinal cord stimulation leads such as the Medtronic® Model 3487A or 3888 leads.

More specifically, the lead 34 may include a plurality of spaced apart electrodes 35 that are adapted to be placed adjacent the spinal cord 32, for example in the intrathecal space or in the epidural space or adjacent the roots of nerves branching off of the spinal cord 32. Although the lead 34 shown in FIG. 1 includes four electrodes 35, an exemplary lead may, e.g., include 2 electrodes to 36 electrodes. For example, the lead 34 may include more than or equal to 2 electrodes, 3 electrodes, 4, electrodes, 5 electrodes, 6 electrodes, 8 electrodes, 10 electrodes, etc., and may include less than or equal to 36 electrodes, 32 electrodes, 28 electrodes, 24 electrodes, 20 electrodes, 18 electrodes, 16 electrodes, 14 electrodes, 12 electrodes, 10 electrodes, 8 electrodes, etc.

Further, although the exemplary system 10 includes one SCS lead 34, the system 10 may include more than one SCS lead (e.g., two SCS leads), each providing a plurality of electrodes configured to be located proximate a patient's spinal cord 32 to deliver therapy (e.g., spinal stimulation) and/or to monitor the patient 12.

In some examples, a programmer 24 may be used to communicate with the IMD 14. The programmer 24 may be a handheld computing device or a computer workstation, which a user, such as a physician, technician, other clinician, and/or patient 12, may use to communicate with the IMD 14. For example, the user may interact with the programmer 24 to retrieve and/or transmit physiological and/or diagnostic information (e.g., one or more intrathoracic impedances, changes in thoracic fluid content, thoracic fluid accumulation values, any other parameters related to a patient's cardiac condition, etc.) from the IMD 14.

The IMD 14 and the programmer 24 may communicate via wireless communication (as represented by the double-headed arrow) using any techniques known in the art. Examples of communication techniques may include, e.g., low frequency or radiofrequency (RF) telemetry, but other techniques are also contemplated.

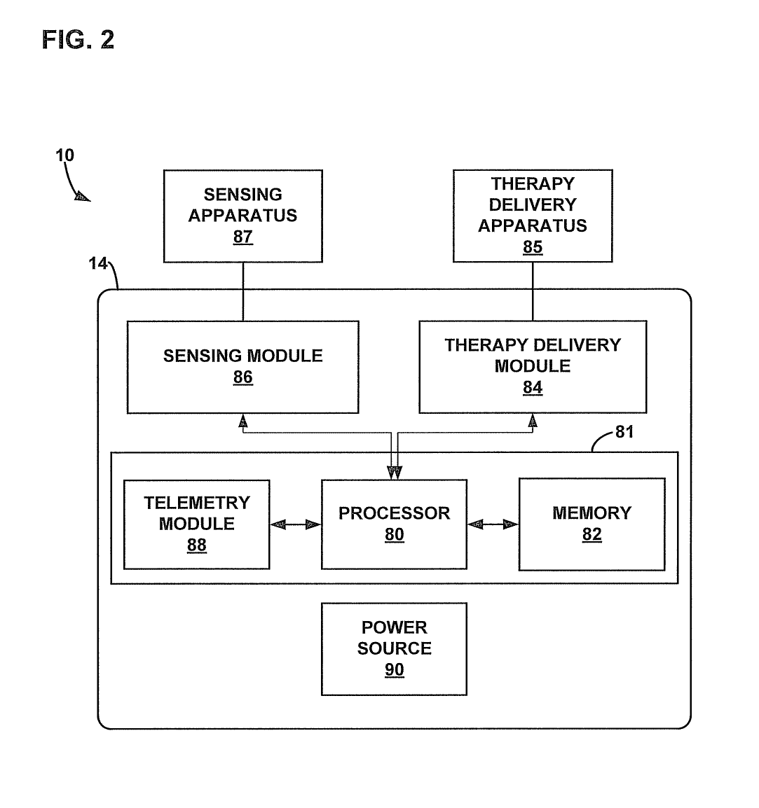

The lead 34 may be electrically coupled to a therapy delivery module (e.g., the lead 34 may be coupled to a SCS pulse generator), a sensing module (e.g., the lead 34 may be coupled to an impedance sensing module), and/or any other modules of the IMD 14 via a connector block 16 (as further described herein with reference to FIG. 2). In some examples, the proximal end of the lead 34 may include electrical contacts that are electrically coupled to respective electrical contacts within the connector block 16 of the IMD 14. In addition, in some examples, the lead 34 may be mechanically coupled to the connector block 16 with the aid of set screws, connection pins, or any other suitable mechanical coupling mechanism.

The lead 34 may include an elongated insulative lead body, which, e.g., may carry a number of concentric coiled conductors, or straight conductors, separated from one another by insulative material. In the illustrated example, the electrodes 35 are located proximate to the distal end of the lead 34. The electrodes 35 may take the form of ring electrodes. Each of the electrodes 35 may be electrically coupled to a respective one of the coiled conductors within the lead body, and thereby coupled to a respective one of the electrical contacts on the proximal end of the lead 34.

A pair of electrodes 35 may further be used by the IMD 14 to measure intrathoracic impedance of the patient 12. For example, a first electrode of the plurality of electrodes 35 may deliver a test, or excitation pulse, and a second electrode (i.e., different from the first) of the plurality of electrodes 35 may measure the voltage difference in the tissue between the first electrode and the second electrode to measure intrathoracic impedance.

Further, for example, a completely independent pair of electrodes (e.g., that may be located in the electromagnetic field created by the test or excitation pulse) may be used to measure the impedance, thus having a quadripolar system. For instance, a test or excitation pulse may be delivered by a first electrode, and impedance may be measured between a second and a third electrode.

The plurality of electrodes 35 may be provided or applied cutaneously or subcutaneously to be located adjacent any of the T1-T12 vertebrae or in any of the C1-C8 locations, and in one or more embodiments, any of the C6-T4 vertebrae, or may be placed adjacent the chest wall. Further, the electrodes 35 may take the form of any of a variety of cutaneous or subcutaneous electrodes. In some embodiments, the electrodes 35 may be disposed immediately adjacent nerve bundles associated with any of the T1-T12 vertebrae.

Conventional subcutaneous electrodes may be surgically inserted into the patient's body. The implantable electrodes may be placed subcutaneously to stimulate underlying muscles, overlying cutaneous nerves, passing somatic nerves, or a combination thereof. For example, various commercially available leads, such as the PISCES, PISCES QUAD PLUS, AND OCTAD model leads, commercially available from Medtronic Corporation, are examples of leads that may be used for this purpose.

As discussed above, subcutaneous electrodes may be carried on leads and inserted near nerve tissue using a delivery device such as a needle. In other instances, subcutaneous electrodes may be carried on the surface of an implanted medical device such as disclosed in commonly assigned U.S. Pat. No. 5,292,336, which is incorporated herein by reference in its entirety. Alternatively, such electrodes may be electrically-isolated from the housing 18 of the IMD 14, as disclosed in commonly-assigned U.S. Pat. No. 5,331,966, which is incorporated herein by reference in its entirety.

In one embodiment, a paddle-type (e.g., flat) lead having a surface area between about 1 square centimeter and about 30 square centimeters or more may be used to accomplish the subcutaneous stimulation. Such a lead may be formed of an insulative material, with programmable electrodes on one or more of the flat sides of the lead. According to this embodiment, the paddle-type lead may be between four and ten millimeters wide so as to be readily passable through a needle such as a twelve-gage needle before it unfolds. In one embodiment, the delivery needle includes an oval or rectangular cross-section of appropriate size to allow for passage of the lead.

In one or more embodiments, the system 10 may include one or more supplemental electrodes other than the SCS electrodes 35 that may be used to measure intrathoracic impedance of the patient 12. For example, the IMD 14 may include one or more housing electrodes 38, which may be formed integrally with an outer surface of a housing 18 (e.g., hermetically-sealed housing) of the IMD 14 or otherwise coupled to the housing 18. Further, for example, an additional implantable device 50 (e.g., a REVEAL cardiac monitor, a subcutaneous implantable cardioverter-defibrillator, an implantable pulse generator, a cardiac resynchronization device, a different monitoring device, a therapy device, etc.) may also include one or more housing electrodes 52, which may be formed integrally with an outer surface of a housing 54 (e.g., hermetically-sealed housing) of the device 50 or otherwise coupled to the housing 54 (e.g., through a lead). Further, the additional device 50 may further include one or more leads carrying electrodes that may be further used to measure intrathoracic impedance of the patient 12.

Further, the supplemental electrodes, such the housing electrodes 38, 52, may be used in conjunction with the SCS electrodes 35 to measure intrathoracic impedance. In other words, the pair of electrodes used in the impedance measurement may include one SCS electrode and one supplemental electrode. For example, at least one supplemental electrode may be used to deliver an excitation pulse for use in impedance measurement while one or more SCS electrodes may measure the intrathoracic impedance. In sum, any combination of the SCS electrodes 35 and the supplemental electrodes (e.g., electrodes 38, 52) may be used in intrathoracic impedance measurements (delivering excitation pulses, measuring voltage, etc.).

The configuration of the therapy system 10 illustrated in FIG. 1 is merely one example. In other examples, a therapy system may include external patch electrodes instead of, or in addition to, the lead 34. Further, in one or more embodiments, the IMD 14 may not be implanted within the patient 12. For example, the IMD 14 may be configured to measure intrathoracic impedance and/or to deliver SCS and other therapies via percutaneous leads that extend through the skin of the patient 12 to a variety of positions within the patient's body and/or via leads that are located on the outside of the patient's body.

FIG. 2 is a functional block diagram of one exemplary configuration of the IMD 14. As shown, the IMD 14 may include a control module 81, a therapy delivery module 84 (e.g., a stimulation generator, or spinal cord stimulator, configured for use in spinal cord stimulation), a sensing module 86, and a power source 90. The control module 81 may include a processor 80, memory 82, and a telemetry module 88. The memory 82 may include computer-readable instructions that, when executed, e.g., by the processor 80, cause the IMD 14 and the control module 81 to perform various functions attributed to the IMD 14 and the control module 81 described herein. Further, the memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, and/or any other digital media.

The processor 80 of the control module 81 may include any one or more of a microprocessor, a controller, a digital signal processor (DSP), an application specific integrated circuit (ASIC), a field-programmable gate array (FPGA), or equivalent discrete or integrated logic circuitry. In some examples, the processor 80 may include multiple components, such as any combination of one or more microprocessors, one or more controllers, one or more DSPs, one or more ASICs, or one or more FPGAs, as well as other discrete or integrated logic circuitry. The functions attributed to the processor 80 herein may be embodied as software, firmware, hardware, or any combination thereof.

The control module 81 is configured to control the therapy delivery module 84 to deliver therapy (e.g., SCS, etc.) to the spinal cord 32 (e.g., initiate the delivery of therapy) according to one or more therapy programs, which may be stored in the memory 82. For example, the control module 81 may control the therapy delivery module 84 to deliver electrical pulses with timings, delays, intervals, amplitudes, pulse widths, and/or frequency, via one or more electrode array configurations specified by the one or more therapy programs.

The therapy delivery module 84 is coupled (e.g., electrically coupled) to therapy delivery apparatus 85. The therapy deliver apparatus 85 may include, among other therapy delivery devices, the electrodes 35 (e.g., via conductors of the lead 34), the housing electrode 38, and the housing electrode 52 (e.g., through wireless communication to the additional device 50). In at least one embodiment, the therapy delivery module 84 may be configured to generate and deliver an electrical test or excitation pulses (e.g., using at least one electrode selected from the SCS electrodes 35, housing electrodes 38, 52, etc.) to the patient's 12 thoracic cavity for use in measuring intrathoracic impedance. For example, therapy delivery module 84 may deliver an electrical test or excitation pulse via one or more of the electrodes 35. Further, in at least one embodiment, the therapy delivery module 84 may be configured to generate and deliver electrical stimulation therapy to the spinal cord 32 (e.g., spinal cord stimulation therapy). For example, therapy delivery module 84 may deliver electrical stimulation via one or more of the electrodes 35.

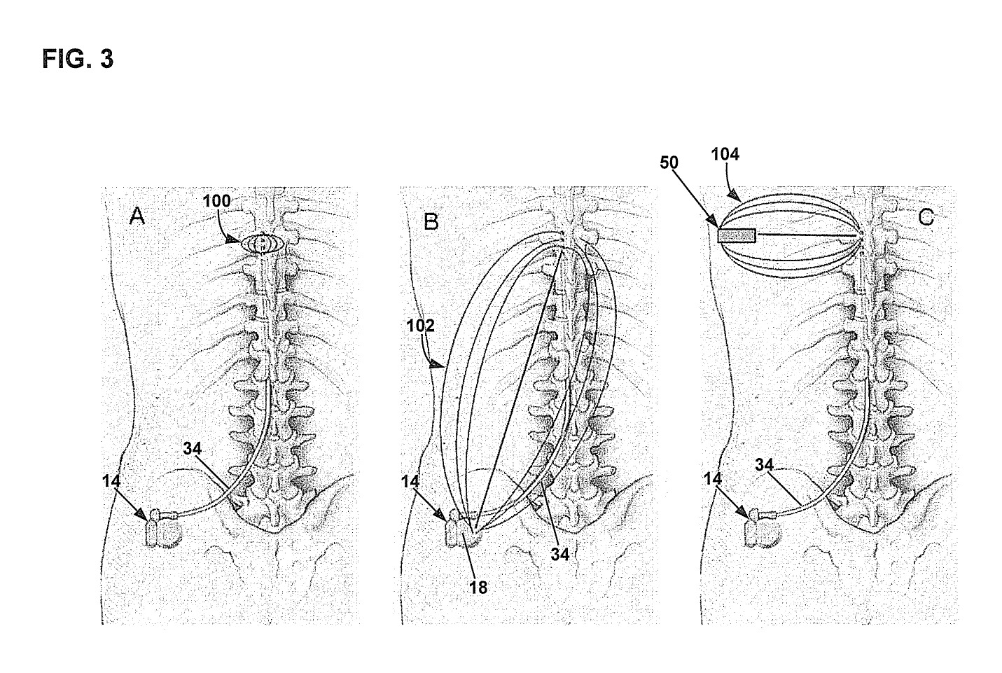

The sensing module 86 is coupled (e.g., electrically coupled, wirelessly coupled, etc.) to the sensing apparatus 87, e.g., to monitor signals from the sensing apparatus 87. The sensing apparatus 87 may include the electrodes 35, 38, and 52, which may be used by the sensing module 86 to measure intrathoracic impedances using various impedance vectors, e.g., such as those impedance vectors shown in FIG. 3.

As used herein, the term “SCS apparatus” may include any devices, modules, and/or apparatus that may be configured to deliver spinal cord stimulation.

The telemetry module 88 of the control module 81 may include any suitable hardware, firmware, software, or any combination thereof for communicating with another device, such as the programmer 24 (see FIG. 1). For example, under the control of the processor 80, the telemetry module 88 may receive downlink telemetry from and send uplink telemetry to the programmer 24 with the aid of an antenna, which may be internal and/or external to the IMD 14. The processor 80 may provide the data to be uplinked to the programmer 24 and the control signals for the telemetry circuit within the telemetry module 88, e.g., via an address/data bus. In some examples, the telemetry module 88 may provide received data to the processor 80 via a multiplexer.

In some examples, the control module 81 (e.g., the processor 80) may transmit measured intrathoracic impedances, intrathoracic impedance values (e.g., intrathoracic impedance composite values, etc.) to the programmer 24, e.g., for cardiac evaluation of the patient (e.g., for use in the treatment of heart failure, for use in identifying changes in thoracic fluid content, for use in the indication of thoracic fluid accumulation, etc.). The programmer 24 may interrogate the IMD 14 to receive such information (e.g., thoracic fluid content or accumulations, etc.).

The various components of the IMD 14 may be further coupled to a power source 90, which may include a rechargeable or non-rechargeable battery. A non-rechargeable battery may be selected to last for several years, while a rechargeable battery may be inductively charged from an external device, e.g., on a daily or weekly basis.

Three different exemplary impedance vector configurations are shown in FIG. 3 that may be used by the exemplary system 10 of FIG. 1 and the exemplary methods described herein. The first impedance vector 100 (depicted in FIG. 3A as magnetic field lines) is generated between at least one pair of SCS electrodes located on lead 34 (e.g., electrodes 35). In other words, the first impedance vector 100 is localized to the region proximate the pair of SCS electrodes on the lead 34.

The second impedance vector 102 (depicted in FIG. 3B as magnetic field lines) is generated between at least one SCS electrode located on lead 34 (e.g., electrodes 35) and at least one housing electrode (e.g., electrode 38) located on the housing 18 of the IMD 14. The third impedance vector 104 (depicted in FIG. 3C as magnetic field lines) is generated between at least one SCS electrode located on lead 34 (e.g., electrodes 35) and at least one electrode (e.g., electrode 52) located on the additional device 50 (e.g., a REVEAL device, a different monitoring device, a therapy device, etc.).

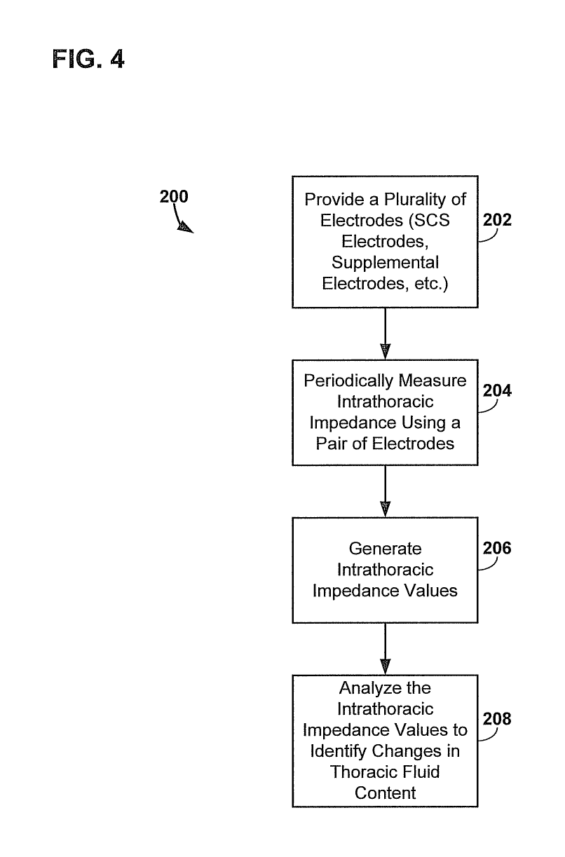

A generalized method 200 for use in an exemplary system (e.g., the exemplary system 10 of FIG. 1) operable for use in cardiac monitoring (e.g., for use in monitoring heart failure) and/or for use in the delivery of SCS (e.g., for providing SCS therapy to treat heart failure) to a patent is diagrammatically depicted in FIG. 4. Method 200 is intended to illustrate the general functional operation of the devices and/or systems described herein, and should not be construed as reflective of a specific form of software or hardware necessary to practice all of the methods described herein. It is believed that the particular form of software will be determined primarily by the particular system architecture employed in the device (e.g., IMD 14) and by the particular detection and therapy delivery methodologies employed by the device and/or system. Providing software and/or hardware to accomplish the described methods in the context of any modern medical device, given the disclosure herein, is within the abilities of one of skill in the art. The method 200 of FIG. 4 includes providing a plurality of electrodes 202 proximate a patient's thoracic cavity. Generally, the electrodes may be classified as spinal cord stimulation (SCS) electrodes (e.g., located along a patient's spinal cord 32) and supplemental electrodes. Exemplary supplemental electrodes may include housing electrodes 38, 52 and any other electrode that may be used to measure intrathoracic impedance. As described herein, SCS electrodes may be applied cutaneously or subcutaneously adjacent any of the T1-T12 vertebrae or in any of the C1-C8 locations. Each SCS electrode may be located in a different location along the patient's spinal cord 32. For example, the SCS electrodes may be spread out or spaced apart along a portion of the patient's spinal cord 32 such that each electrode may affect the patient's spinal cord differently than each of the other electrodes. The supplemental electrodes may be located anywhere in the patient's body such that they may be used to measure intrathoracic impedance. In at least one embodiment, at least one supplemental electrode is located proximate the patient's thoracic cavity (e.g., proximate the patient's abdomen, beneath the skin in the patient's chest but outside the thoracic cavity, etc.).

Using at least one pair of the electrodes, intrathoracic impedance of a patient may be periodically monitored 204. In at least one embodiment, intrathoracic impedance may be measured periodically over an extended period of time. The extended period of time may be greater than one day and may extend to multiple days, months, and/or years. In other words, in at least one embodiment, the extended period of time through which intrathoracic impedance may be periodically monitored 204 is a time period having any length greater than a day, e.g., in order to observe changes in trends of the impedance that might be associated with acutely worsening heart failure as well as patterns of diurnal variation.

Patients with a history of chronic heart failure (CHF) have a significantly greater daily circadian variation (CV) in intrathoracic impedance than patients without CHF history. In other words, changes in intrathoracic impedance may correlate with changes in pulmonary capillary wedge pressure during acute diuresis and may predict heart failure hospitalization. Further, cavitary fluid shifts associated with posture may also result in a CV in intrathoracic impedance. As such, in one or more embodiments, intrathoracic impedance of a patient may be periodically monitored 204 to analyze daily CV in intrathoracic impedance (see, e.g., U.S. Pat. App. Pub. No. 2007/0156061 A1 filed Dec. 30, 2005 to Michael Hess, which is incorporated by reference herein in its entirety). For example, intrathoracic impedance may be measured for 4 time intervals throughout an entire day such as, e.g., 6:00 AM to 12:00 PM, 12:00 PM to 6:00 PM, 6:00 PM to 12:00 AM, and 12:00 AM to 6:00 AM. The mean impedance of the two day time intervals (e.g., 6:00 AM to 12:00 PM and 12:00 PM to 6:00 PM) may be subtracted from the mean impedance of the two night-time intervals (6:00 PM to 12:00 AM, and 12:00 AM to 6:00 AM) in order to determine the daily circadian variation in intrathoracic impedance, which may be used in cardiac monitoring. For example, patients having greater circadian variation in intrathoracic impedance may have a greater likelihood of heart failure. See, e.g., U.S. Pat. App. Pub. No. 2007/0156061 A1 filed Dec. 30, 2005 to Michael Hess.

In at least one embodiment, to periodically measure intrathoracic impedance using one or more pairs of electrodes 204, the exemplary method 200 may measure intrathoracic impedance a selected number of times throughout each entire day. For example, intrathoracic impedance may be measured using at least one pair of electrodes 24 times throughout each entire day (i.e., hourly). In other words, periodically measuring intrathoracic impedance 204 may be non-continuous and may include individual measurements scheduled apart from one another over the course of the extended time period.

Instead of periodically measuring intrathoracic impedance based on time periods, intrathoracic impedance may be measured based on cardiac cycles. For example, intrathoracic impedance may be measured using at least one pair of electrodes once per cardiac cycle (e.g., such that changes in impedance within the cardiac cycle are not observed). In other embodiments, intrathoracic impedance may be measured less than once per cardiac cycle, e.g., once every 2 cardiac cycles, once every 5 cardiac cycles, once every 10 cardiac cycles, once every 20 cardiac cycles, etc.

Further, in at least embodiment, to periodically measure intrathoracic impedance using one or more pairs of electrodes 204, the exemplary method 200 may measure intrathoracic impedance using multiple pairs of electrodes. For example, instead of only taking a single impedance intrathoracic impedance measurement, multiple impedance measurements may be taken using multiple pairs of electrodes. Further, the multiple impedance measurements may be taken simultaneously (e.g., at the same time, or near the same time so as to not overlap) or non-simultaneously (e.g., scheduled at different times of the entire day).

The method 200 may further include generating intrathoracic impedance values of the patient based on the measured intrathoracic impedance 206. More specifically, these intrathoracic impedance values may be generated for selected time periods during the extended period of time.

For example, the selected time periods could be days. In essence, one intrathoracic impedance value could be generated based on all the periodically measured values for an entire day. More specifically, each of the periodically measured values for the entire day may be generated using any statistical an mathematical process including computing trends, slopes, means, medians, and/or weighted averages, excluding outliers and/or anomalies, etc. In other words, in at least one embodiment, the method 200 may generate an intrathoracic impedance composite value based at least one intrathoracic impedance measurement taken throughout each entire day.

Although the exemplary selected time period provide may be a day, the selected time period may be less than a day (e.g., 6 hours, etc.) or greater than a day (e.g., two days, one week, etc.). Further, an intrathoracic impedance composite value based on at least one intrathoracic impedance measurement taken throughout each entire selected time period may be generated. For example, if the selected time period is 6 hours, an intrathoracic impedance composite value may be generated every 6 hours. Likewise, if the selected time period is 1 week, an intrathoracic impedance composite value may be generated every week.

Changes in the intrathoracic impedance values generated in process 206 may be useful for cardiac monitoring, e.g., monitoring thoracic fluid content, which may be useful to indicate heart failure. The method 200 may further include analyzing the intrathoracic impedance values generated for the selected time periods over the extended period of time to identify changes in the patient's thoracic fluid content for use in cardiac monitoring 208. As described herein, the extended time period may be a time period greater than a day, e.g., weeks or months. By analyzing the generated intrathoracic impedance values over the course of the extended time period, e.g., a month, changes in thoracic fluid content may be identified (e.g., determined or indicated) as they occur, which may be useful to indicate heart failure. For example, a presently generated intrathoracic impedance value may be significantly different than a rolling average of the past five generated intrathoracic impedance values, which may indicate a cardiac issue such as heart failure.

As described herein, in at least one embodiment, an intrathoracic impedance composite value may be generated for each day (e.g., in the instance where the selected time period is a day). The method 200 may analyze each of the generated intrathoracic impedance composite values over the course of the extended time period (e.g., each time an intrathoracic impedance composite value is generated, it may be analyzed). In other words, in at least one embodiment, the patient's fluid content (e.g., indicated by the intrathoracic impedance composite value) may be analyzed each day in view of the patient's fluid content over the extended time period.

In at least one embodiment, the generated impedance values may be analyzed in view of a baseline value, which may indicate acceptable thoracic fluid content. Further, the baseline value may be at least partially based on previously generated intrathoracic impedance values.

In at least another embodiment, the trend, or pattern, of the generated impedance values may be analyzed in view of a baseline value, and the baseline value may be indicative of acceptable change from one selected time period (e.g., one day) to another (see, e.g., U.S. Pat. App. Pub. No. 2009/0275854 A1 filed Apr. 30, 2008 to Zielinski et al, which is incorporated by reference herein in its entirety). For example, a significant day-to-day change may be indicative of heart failure.

The exemplary systems and methods described in U.S. Pat. No. 7,986,994 to Stadler et al. issued Jul. 26, 2011 and U.S. Pat. App. Pub. No. 2010/0030292 A1 to Sarkar et al. filed on Jul. 31, 2008, each of which are incorporated by reference herein in their entireties, may be used in conjunction with the systems and methods for use in detecting changes in intrathoracic impedance described herein.

Further, in at least one embodiment, the real and reactive components of the measured intrathoracic impedances may be analyzed, e.g., in order to difference the relative contribution to the measured impedance chance of the relatively more resistive fluid component and the relatively more reactive tissue component (see, e.g., U.S. Pat. No. 5,282,840 issued on Feb. 1, 1994 to Terrence Hudrlik, U.S. Pat. No. 5,454,377 issued on Oct. 3, 1995 to Dzwonczyk, et al., and U.S. Pat. No. 6,473640 issued on Oct. 29, 2002 to Jay Erlebacher, each of which are incorporated herein by reference in their entireties.

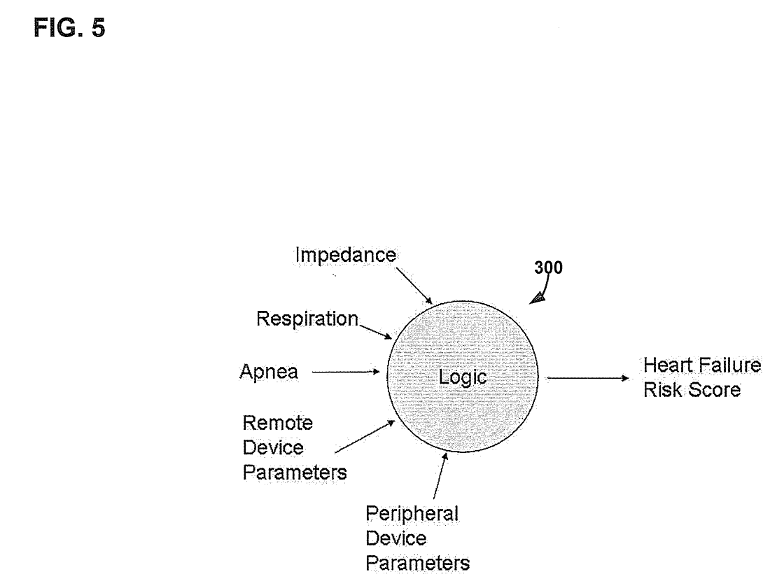

The impedance values generated and/or the analysis of the exemplary method 200 described herein with reference to FIG. 4 may be further utilized as at least one component in a method of providing a heart failure risk score 300 as depicted in FIG. 5. In essence, the intrathoracic impedance measurements and/or intrathoracic impedance analysis could be combined with other measurements performed by peripheral and/or other implanted devices using established artificial intelligence techniques to determine a dynamic heart failure risk score. This may be referred to as an integrated diagnostics approach to improve the clinical utility of heart failure parameters monitored by an implantable spinal cord stimulator.

For example, one or more physiological parameters may be monitored, such as intrathoracic impedance, respiration, apnea, blood pressure, weight, blood glucose, brain natriuretic peptide (BNP), international normalized ratio (INR), temperature, patient-reported symptoms (e.g., dyspnea, insomnia, fatigue, depression, etc.), etc. using implantable medical devices, remote devices, peripheral devices (e.g., scales, blood pressure cuffs, etc.), etc. These one or more physiological parameters may be interpreted by an artificial intelligence model (e.g., a Bayesian network model) to produce a dynamic daily heart failure risk score. The daily heart failure risk score may indicate to a patient whether he/she needs to seek immediate medical attention.

As described, respiration, e.g., more specifically, respiratory disturbances, may be used in conjunction with the intrathoracic impedance measurements and/or intrathoracic impedance analysis to determine a dynamic heart failure score (see, e.g., U.S. Pat. No. 7,160,252 issued Jan. 9, 2007 to Cho, et al.). Further, as described, blood pressure, e.g., more specifically pulsatile signals in blood pressure, may be used in conjunction with the intrathoracic impedance measurements and/or intrathoracic impedance analysis to determine a dynamic heart failure score.

In at least one embodiment, a computing system may receive the one or more physiological parameters and, based on such received parameters, may compute a heart failure risk score. Many of the one or more physiological parameters (e.g., patient weight, etc.) may be measured and inputted by the patient or a clinician monitoring the patient (e.g., directly into the computing system, using another computing device, via the internet, etc.). Further, many of the one or more physiological parameters may be transmitted and inputted directly from the remote, implantable, and/or non-implantable medical devices that may be monitoring the patient (e.g., configured to monitor the patient's intrathoracic impedance values of an extended period of time, etc.).

All patents, patent documents, and references cited herein are incorporated in their entirety as if each were incorporated separately. This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

What is claimed:

1. A system for use in cardiac monitoring comprising:
spinal cord stimulation (SCS) apparatus, wherein the SCS apparatus comprises a plurality of SCS electrodes locatable along a patient's spinal cord; and
a control module coupled to the SCS apparatus and configured to:
periodically measure intrathoracic impedance using one or more pairs of SCS electrodes selected from the plurality of SCS electrodes over an extended period of time,
generate, for selected time periods during the extended period of time, intrathoracic impedance values of the patient based on the periodically measured intrathoracic impedance, and
analyze the intrathoracic impedance values generated for the selected time periods over the extended period of time to identify changes in the patient's thoracic fluid content for use in cardiac monitoring.

2. The system of claim 1, wherein, to periodically measure intrathoracic impedance using one or more pairs of SCS electrodes, the control module is further configured to measure intrathoracic impedance a selected number of times throughout each entire day, and
wherein, to generate intrathoracic impedance values of the patient based on the periodically measured intrathoracic impedance, the control module is further configured to generate an intrathoracic impedance composite value based at least on multiple intrathoracic impedance measurements taken throughout each entire day.

3. The system of claim 2, wherein, to analyze the intrathoracic impedance values generated for the selected time periods over the extended period of time, the control module is further configured to analyze the intrathoracic impedance composite value generated for each day over the extended period of time, wherein the extended period of time is greater than one day.

4. The system of claim 1, wherein, to analyze the intrathoracic impedance values generated for the selected time periods over the extended period of time, the control module is further configured to compare an intrathoracic impedance value for a current selected time period to a baseline value, wherein the baseline value is at least based on previously generated intrathoracic impedance values.

5. The system of claim 1, wherein, to periodically measure intrathoracic impedance using one or more pairs of SCS electrodes, the control module is further configured to simultaneously measure intrathoracic impedance a selected number of times throughout each entire day using multiple pairs of SCS electrodes.

6. A system for use in cardiac monitoring comprising:
a plurality of SCS electrodes locatable along a patient's spinal cord;
at least one supplemental electrode locatable proximate the patient's thoracic cavity; and
a control module coupled to the SCS electrodes and the at least one supplemental electrode, wherein the control module is configured to:
periodically measure intrathoracic impedance using one or more pairs of electrodes over an extended period of time, wherein each pair of electrodes comprises:
an SCS electrode selected from the plurality of SCS electrodes, and
an electrode selected from the at least one supplemental electrode,
generate, for selected time periods during the extended period of time, intrathoracic impedance values of the patient based on the periodically measured intrathoracic impedance, and
analyze the intrathoracic impedance values generated for the selected time periods over the extended period of time to identify changes in the patient's thoracic fluid content for use in cardiac monitoring.

7. The system of claim 6, wherein, to periodically measure intrathoracic impedance using one or more pairs of electrodes, the control module is further configured to measure intrathoracic impedance a selected number of times throughout each entire day, and
wherein, to generate intrathoracic impedance values of the patient based on the periodically measured intrathoracic impedance, the control module is further configured to generate an intrathoracic impedance composite value based at least on multiple intrathoracic impedance measurements taken throughout each entire day.

8. The system of claim 7, wherein, to analyze the intrathoracic impedance values generated for the selected time periods over the extended period of time, the control module is further configured to analyze the intrathoracic impedance composite value generated for each day over the extended period of time, wherein the extended period of time is greater than one day.

9. The system of claim 6, wherein, to analyze the intrathoracic impedance values generated for the selected time periods over the extended period of time, the control module is further configured to compare an intrathoracic impedance value for a current selected time period to a baseline value, wherein the baseline value is at least based on previously generated intrathoracic impedance values.

10. The system of claim 6, wherein, to periodically measure intrathoracic impedance using one or more pairs of electrodes, the control module is configured to simultaneously measure intrathoracic impedance a selected number of times throughout each entire day using multiple pairs of electrodes.

11. The system of claim 6, wherein the system further comprises an implantable medical device comprising a housing, and wherein the at least one supplemental electrode is located on the housing of the implantable medical device.

12. The system of claim 11, wherein the implantable medical device is a SCS stimulator for delivering stimulation pulses to one or more of the plurality of SCS electrodes.

13. The system of claim 11, wherein the implantable medical device is a different implantable medical device separate from an SCS stimulator for delivering stimulation pulses to one or more of the plurality of SCS electrodes.

14. A method for use in cardiac monitoring using spinal cord stimulation (SCS) apparatus:
providing a plurality of SCS electrodes located along a patient's spinal cord;
periodically measuring intrathoracic impedance using one or more pairs of SCS electrodes selected from the plurality of SCS electrodes over an extended period of time;
generating, for selected time periods during the extended period of time, intrathoracic impedance values of the patient based on the periodically measured intrathoracic impedance; and
analyzing the intrathoracic impedance values generated for the selected time periods over the extended period of time to identify changes in the patient's thoracic fluid content for use in cardiac monitoring.

15. The method of claim 14, wherein periodically measuring intrathoracic impedance using one or more pairs of SCS electrodes comprises measuring intrathoracic impedance a selected number of times throughout each entire day, and
wherein generating intrathoracic impedance values of the patient based on the periodically measured intrathoracic impedance comprises generating an intrathoracic impedance composite value based at least on multiple intrathoracic impedance measurements taken throughout each entire day.

16. The method of claim 15, wherein analyzing the intrathoracic impedance values generated for the selected time periods over the extended period of time comprises analyzing the intrathoracic impedance composite value generated for each day over the extended period of time, wherein the extended period of time is greater than one day.

17. The method of claim 14, wherein analyzing the intrathoracic impedance values generated for the selected time periods over the extended period of time comprises comparing an intrathoracic impedance value for a current selected time period to a baseline value, wherein the baseline value is at least based on previously generated intrathoracic impedance values.

18. The method of claim 14, wherein periodically measuring intrathoracic impedance using one or more pairs of SCS electrodes comprises simultaneously measuring intrathoracic impedance a selected number of times throughout each entire day using multiple pairs of SCS electrodes.

19. A method for use in cardiac monitoring using spinal cord stimulation (SCS) apparatus:
providing a plurality of SCS electrodes located along a patient's spinal cord;
providing at least one supplemental electrode located proximate the patient's thoracic cavity;
periodically measuring intrathoracic impedance using one or more pairs of electrodes over an extended period of time, wherein each pair of electrodes comprises:
an SCS electrode selected from the plurality of SCS electrodes, and
an electrode selected from the at least one supplemental electrode;
generating, for selected time periods during the extended period of time, intrathoracic impedance values of the patient based on the periodically measured intrathoracic impedance; and
analyzing the intrathoracic impedance values generated for the selected time periods over the extended period of time to identify changes in the patient's thoracic fluid content for use in cardiac monitoring.

20. The method of claim 19, wherein periodically measuring intrathoracic impedance using one or more pairs of electrodes comprises measuring intrathoracic impedance a selected number of times throughout each entire day, and
wherein generating intrathoracic impedance values of the patient based on the periodically measured intrathoracic impedance comprises generating an intrathoracic impedance composite value based at least on multiple intrathoracic impedance measurements taken throughout each entire day.

21. The method of claim 20, wherein analyzing the intrathoracic impedance values generated for the selected time periods over the extended period of time comprises analyzing the intrathoracic impedance composite value generated for each day over the extended period of time, wherein the extended period of time is greater than one day.

22. The method of claim 19, wherein analyzing the intrathoracic impedance values generated for the selected time periods over the extended period of time comprises comparing an intrathoracic impedance value for a current selected time period to a baseline value, wherein the baseline value is at least based on previously generated intrathoracic impedance values.

23. The method of claim 19, wherein periodically measuring intrathoracic impedance using one or more pairs of electrodes comprises simultaneously measuring intrathoracic impedance a selected number of times throughout each entire day using multiple pairs of electrodes.

24. The method of claim 19, wherein the method further comprises providing an implantable medical device comprising a housing, and wherein the at least one supplemental electrode is located on the housing of the implantable medical device.

25. The method of claim 24, wherein the implantable medical device is a SCS stimulator for delivering stimulation pulses to one or more of the plurality of SCS electrodes.

26. The method of claim 25, wherein the implantable medical device is a different implantable medical device separate from an SCS stimulator for delivering stimulation pulses to one or more of the plurality of SCS electrodes.
